(19) United States
(12) Patent Application Publication
(10) Pub. No.: US 2026/0334952 A1
(43) Pub. Date: Oct. 29, 2026

(54) Intestinal Postoperative Anti-Fistula Device

(71) Applicant: The Second Hospital of Hebei Medical University, Shijiazhuang (CN)

(72) Inventors: Guiying Wang, Shijiazhuang (CN); Zesong Meng, Shijiazhuang (CN); Baokun Li, Shijiazhuang (CN)

(21) Appl. No.: 19/630,209

(22) Filed: Mar. 26, 2026

(30) Foreign Application Priority Data
Apr. 25, 2025 (CN) 202510533167.2

(51) Int. Cl. A61B 17/11; A61B 17/00; A61B 17/06

(52) U.S. Cl. CPC A61B 17/1114; A61B 2017/00004; A61B 2017/06176; A61B 2017/1132

(57) ABSTRACT

An intestinal postoperative anti-fistula device includes an anti-fistula membrane assembly and a degradable proximal ring. The anti-fistula membrane assembly includes a membrane tube. The first end of the membrane tube is provided with a storage cavity, which is formed by folding back the first end of the membrane tube and provided with multiple degradation through-holes. The degradable proximal ring is arranged in the storage cavity and configured to fix the membrane tube to an intestine by suturing. The second end of the membrane tube is provided with a distal support ring that is configured to provide support for the second end of the membrane tube and guide the membrane tube to axially compress and extend.

### CROSS-REFERENCE TO RELATED APPLICATIONS

This application claims priority to Chinese Patent Application No. CN 202510533167.2, filed on Apr. 25, 2025 and entitled “intestinal postoperative anti-fistula device”. The disclosure of the aforementioned application is hereby incorporated herein by reference in its entirety.

### TECHNICAL FIELD

The present application relates to the technical field of medical devices, particularly to an anti-fistula device, and more specifically to an intestinal postoperative anti-fistula device.

### BACKGROUND

Preventing fistula occurrence following intestinal surgery is an important nursing and therapeutic goal. A fistula refers to the leakage of intestinal content through an abnormal channel into a body cavity or outside the body, which can easily lead to serious complications such as infection and malnutrition. Currently, the incidence of anastomotic fistula following small intestine surgery is approximately 3%-5%, which is low. However, the incidence of anastomotic fistula following colon surgery is typically between 5% and 15%. In particular, for low rectal cancer surgery, due to the lower surgical site, the risk of anastomotic fistula is high, with an incidence rate of about 10%-20%.

An intestinal postoperative anti-fistula device is a medical device used to prevent anastomotic fistula following intestinal surgery. It is used in intestinal anastomosis procedures, aiming to reinforce the anastomosis, reduce the risk of intestinal content leakage, and thereby lower the incidence of anastomotic fistula.

A Chinese patent titled “An Anastomotic Anti-Fistula Device, Sealing Storage Box and Loader” (Publication No. CN113520704B) discloses an anastomotic anti-fistula technology. It solves the problem that existing large intestine anti-fistula devices need to be retrieved, which increases surgical complexity and patient suffering, by configuring the stent woven body to be made of fully degradable material with self-expanding properties and also configuring the membrane covering the surface of said stent woven body to be made of fully degradable material. However, the structure of the device, which combines the stent woven body and the membrane, is complex, resulting in high production and usage costs. Furthermore, because the membrane envelops the surface of the stent woven body, intestinal content comes into direct contact with the mesh structure of the stent woven body, resulting in high resistance for intestinal content to pass through the stent woven body. This can easily cause premature detachment of the anti-fistula device, increasing postoperative risks. Therefore, an intestinal postoperative anti-fistula device is proposed by the present application.

### SUMMARY

This and other problems are generally solved or circumvented, and technical advantages are generally achieved, by embodiments of the present application which provide intestinal postoperative anti-fistula devices.

### Technical Problems

The present application provides intestinal postoperative anti-fistula devices to solve or alleviate technical problems existing in the prior art, at least providing beneficial alternatives.

### Technical Solutions

An intestinal postoperative anti-fistula device provided in the present application includes an anti-fistula membrane assembly and a degradable proximal ring. The anti-fistula membrane assembly includes a membrane tube.

The first end of the membrane tube is provided with a storage cavity that is formed by a folding section at the first end of the membrane tube. The degradable proximal ring is arranged in the storage cavity. The degradable proximal ring is configured to fix the membrane tube to an intestine by suturing. A first wall of the storage cavity is provided with a plurality of degradation through-holes uniformly distributed.

The second end of the membrane tube, which is away from the storage cavity, is provided with a distal support ring configured to provide support for the second end of the membrane tube and guide the membrane tube to axially compress and extend.

Optionally, the distal support ring includes three first segments and three second segments arranged alternately and connected end-to-end to form a complete circular ring, where the second end of the membrane tube is fixedly connected to one side of the three first segments and the three second segments.

Optionally, the thickness of each first segment is 2 mm, and the thickness of each second segment is 1 mm.

The intestinal postoperative anti-fistula device provided by the present application at least has the following advantages.

When the anti-fistula device is in use, before intestinal anastomosis, the degradable proximal ring is sutured to a proximal intestine, thereby fixing both the degradable proximal ring and the membrane tube to the proximal intestine. After the anastomosis, the distal support ring is pulled towards a distal intestine connected to the proximal intestine, such that the distal support ring guides the membrane tube to extend axially to completely cover the anastomosis, while the distal support ring supports the membrane tube. Therefore, intestinal content is isolated from the anastomosis and they cannot come into contact, fully protecting the anastomosis. Furthermore, the interior of the anti-fistula device forms a smooth passage overall, reducing the resistance to intestinal content and effectively lowering the risk of premature detachment.

The degradable proximal ring is confined in the storage cavity, which is formed by folding back one end edge of the membrane tube and provided with the degradation through-holes, allowing the degradable proximal ring to be degraded in the storage cavity, while the distal support ring is made of non-degradable materials, making the degradation and detachment time of the anti-fistula device more stable. Moreover, the overall structure of the anti-fistula device is simple, effectively reducing production and usage costs.

The above technical solution is for illustrative purposes only and is not intended to be limiting. Further embodiments and features of the present application will be detailed below with reference to the drawings.

### BRIEF DESCRIPTION OF THE DRAWINGS

To describe the technical solutions in the embodiments of the present application or the prior art more clearly, the following briefly introduces the drawings required for description. Obviously, the drawings in the following description show only some embodiments of the present application. For those skilled in the art, other drawings may be obtained based on these drawings without inventive effort.

FIG. 1 is a schematic perspective view of an intestinal postoperative anti-fistula device according to embodiments of the present application;

FIG. 2 is a schematic longitudinal cross-sectional view of the first end of an intestinal postoperative anti-fistula device according to embodiments of the present application;

FIG. 3 is a view of FIG. 2 with the degradable proximal ring omitted;

FIG. 4 is a schematic side view of a distal support ring according to embodiments of the present application;

FIG. 5 is a schematic top view of a distal support ring according to embodiments of the present application;

FIG. 6 is a schematic top view of a degradable proximal ring according to embodiment 1 of the present application;

FIG. 7 is a schematic side view of a degradable proximal ring according to embodiment 1 of the present application;

FIG. 8 is a schematic top view of a degradable proximal ring according to embodiment 2 of the present application;

FIG. 9 is a schematic side view of a degradable proximal ring according to embodiment 2 of the present application; and

FIGS. 10(a)-(d) show an exemplary usage process of an intestinal postoperative anti-fistula device according to embodiments of the present application.

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION OF ILLUSTRATIVE EMBODIMENTS

In the following description, only certain exemplary embodiments are described. As those skilled in the art will recognize, the described embodiments may be modified in various ways without departing from the spirit or scope of the present application. Accordingly, the drawings and description are to be regarded as illustrative in nature and not restrictive.

It should be noted that terms “first” and “second” are used only for distinguishing features, and should not be construed as indicating or implying relative importance or implicitly specifying the quantity of the indicated technical features.

The embodiments of the present application are described in detail below with reference to the drawings.

### Embodiment 1

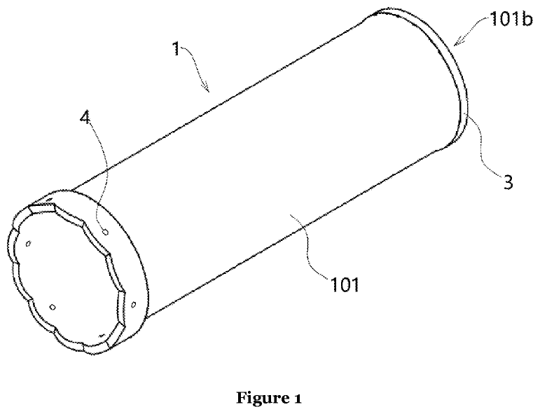

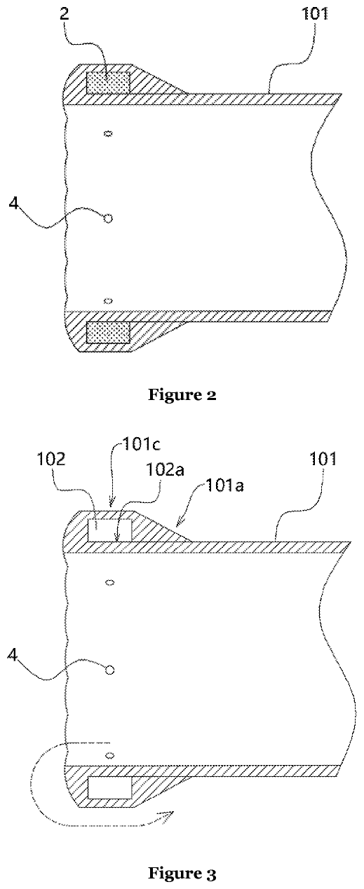

In Embodiment 1, referring to FIGS. 1 to 3, the intestinal postoperative anti-fistula device provided by the present application includes an anti-fistula membrane assembly 1 and a degradable proximal ring 2. The anti-fistula membrane assembly 1 includes a membrane tube 101 and a storage cavity 102.

The storage cavity 102 is provided at a first end 101a of the membrane tube 101. The storage cavity 102 is formed by a folding section 101c at a first end 101a of the membrane tube 101. The first end 101a of the membrane tube 101 is folded back onto itself and secured to enclose the storage cavity 102, as indicated in FIG. 3 by the dashed arrow. The degradable proximal ring 2 is arranged in the storage cavity 102. A distal support ring 3 is fixedly connected to the second end 101b of the membrane tube 101 away from the storage cavity 102. A first wall 102a of the storage cavity 102 is provided with a plurality of degradation through-holes 4 uniformly distributed.

The degradable proximal ring 2 is used to fix the membrane tube 101 to an intestine by suturing.

The distal support ring 3 is used to provide support for the second end 101b of the membrane tube 101 and guide the membrane tube 101 to axially compress and extend. Optionally, the membrane tube 101 is made of polymeric membrane, and it can be compressed and extended along the extension direction of an intestine.

In the present application, the terms “proximal” and “distal” are used to distinguish between two positions/ends distributed along an intestine. Based on the normal physiological flow direction of intestinal content as a reference, “proximal” refers to the position/end closer to the starting point of the physiological flow (mouth/stomach), and “distal” refers to the position/end closer to the endpoint of the physiological flow (anus).

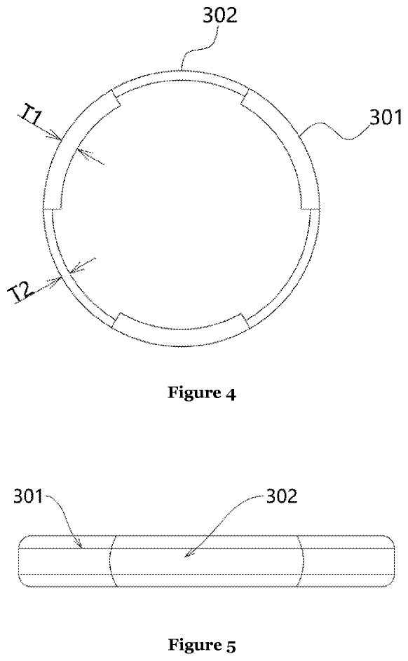

In an example, referring to FIGS. 4 and 5, the distal support ring 3 includes three first segments 301 and three second segments 302.

The three first segments 301 and three second segments 302 are arranged alternately and connected end-to-end to form a complete circular ring. The second end 101b of the membrane tube 101 is fixedly connected to one side of the three first segments 301 and second segments 302. The thickness T1 of the first segment 301 is 2 millimeters (mm), and the thickness T2 of the second segment 302 is 1 mm.

By using the three first segments 301 and three second segments 302 to form a ring, support is provided for one end of the membrane tube 101, enabling the membrane tube 101 to effectively fit against the wall of an intestine, and facilitating the movement (e.g., extension) of the membrane tube 101 by moving the distal support ring 3 externally from the intestine.

Furthermore, as the thickness of the second segment 302 is less than that of the first segment 301, the second segments 302 are more prone to bending, which facilitates the passage of the distal support ring 3 through the anus for expulsion from the body.

In an example, the membrane tube 101 and the degradable proximal ring 2 are made of radiopaque degradable composite material (e.g., polyglycolic acid (PGA) and barium sulfate composite material) with elasticity. The degradation and detachment time of the anti-fistula membrane assembly 1 and the degradable proximal ring 2 is 10 to 14 days. The surfaces of the membrane tube 101 may be coated with lubricant.

The membrane tube 101 can protect the anastomosis before it heals. By using the elastic radiopaque degradable composite material to fabricate the degradable proximal ring 2, after the anastomosis heals, the degradable proximal ring 2 can degrade, causing the membrane tube 101 to detach.

Optionally, the inner surface of the membrane tube 101 may be coated with lubricant to reduce the resistance of intestinal content passing through the membrane tube 101.

Optionally, the outer surface of the membrane tube 101 may be coated with lubricant to reduce friction between the membrane tube 101 and the inner wall of the intestine.

The in vitro degradation test results for the degradable proximal ring 2 are shown in the following table:

| Date (MM/DD/YYYY) | 12/17/2023 | 12/24/2023 | 12/30/2023 | 01/16/2024 | 01/23/2024 | 01/30/2024 |
|---|---|---|---|---|---|---|
| Test Period | 0 | 7 | 12 | 21 | 28 | 35 |
| Material | PGA and barium sulfate composite material | | | | | |
| Strength (N) | 54.153 | 48.147 | 42.509 | 28.057 | 11.932 | 5.223 |
| Degradation Retention Rate | 100% | 88.91% | 78.50% | 51.81% | 22.03% | 9.64% |

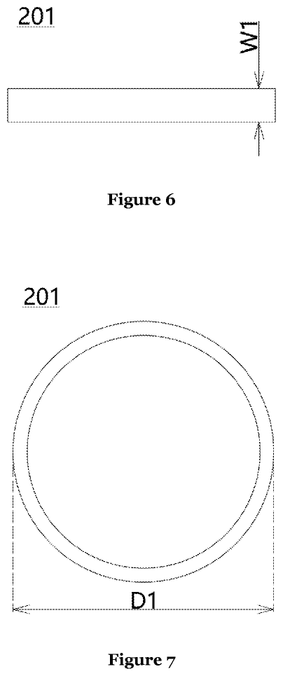

In an example, the degradable proximal ring 2 and an intestine are sutured using barbed suture. Referring to FIGS. 6 and 7, the degradable proximal ring 2 may be a circular degradable ring 201 hemmed in by the walls of the storage cavity 102. The outer diameter D1 of the circular degradable ring 201 may be 33 mm, and its width W1 may be 3 mm.

By folding one end edge of the membrane tube 101, the storage cavity 102 is formed at the end of the membrane tube 101. The storage cavity 102 limits the circular degradable ring 201 in position. The circular degradable ring 201 is fixed to the intestine via barbed sutures, thereby ensuring that the anti-fistula device remains in a predetermined position during the healing of the anastomosis. Throughout the anastomotic healing process, the membrane tube 101 isolates the intestinal content from the anastomotic site, thus providing protection and reducing the incidence of fistula formation. Additionally, during healing, microorganisms from the intestinal lumen can enter the storage cavity 102 through the degradation through-holes 4 and come into contact with the circular degradable ring 201, causing the circular degradable ring 201 to gradually degrade over a preset period. Once degradation reaches a certain level, the anti-fistula device detaches from the intestine and is excreted from the body.

In an example, the intestinal postoperative anti-fistula device provided in Embodiment 1 is applicable for fistula prevention following sphincter-preserving surgery for low or ultra-low rectal cancer.

### Embodiment 2

In Embodiment 2, referring to FIGS. 1 to 3, as in Embodiment 1, the intestinal postoperative anti-fistula device provided by the present application includes an anti-fistula membrane assembly 1 and a degradable proximal ring 2. The anti-fistula membrane assembly 1 includes a membrane tube 101 and a storage cavity 102.

The storage cavity 102 is provided at a first end 101a of the membrane tube 101. The storage cavity 102 is formed by a folding section 101c at a first end 101a of the membrane tube 101. The degradable proximal ring 2 is arranged in the storage cavity 102. A distal support ring 3 is fixedly connected to the second end 101b of the membrane tube 101 away from the storage cavity 102. A first wall 102a of the storage cavity 102 is provided with a plurality of degradation through-holes 4 uniformly distributed.

The degradable proximal ring 2 is used to fix the membrane tube 101 to an intestine by suturing.

The distal support ring 3 is used to provide support for the second end 101b of the membrane tube 101 and guide the membrane tube 101 to axially compress and extend.

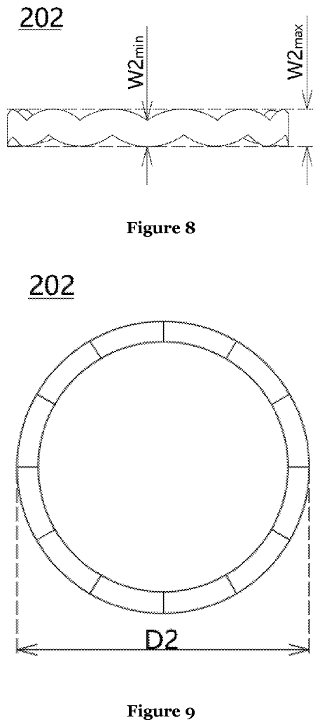

Other contents identical to Embodiment 1 will not be repeated here. The difference from Embodiment 1 is that in Embodiment 2, referring to FIGS. 8 and 9, the degradable proximal ring 2 is a wave-shaped degradable ring 202 hemmed in by the walls of the storage cavity 102. The outer diameter D2 of the wave-shaped degradable ring 202 may be 33 mm, its maximum width W2max may be 3.5 mm, and its minimum width W2 min may be 3 mm.

In Embodiment 2, the wave-shaped degradable ring 202 replaces the circular degradable ring 201. The minimum width of the wave-shaped degradable ring 202 may be set to not less than the width of the circular degradable ring 201, therefore the wave-shaped degradable ring 202 has a larger support area. Additionally, the irregular surface of the wave-shaped degradable ring 202 can increase friction, which is beneficial for its relative fixation to the intestine and makes suturing operation more convenient.

In an example, the intestinal postoperative anti-fistula device provided in Embodiment 2 is applicable for fistula prevention following sphincter-preserving surgery for low or ultra-low rectal cancer.

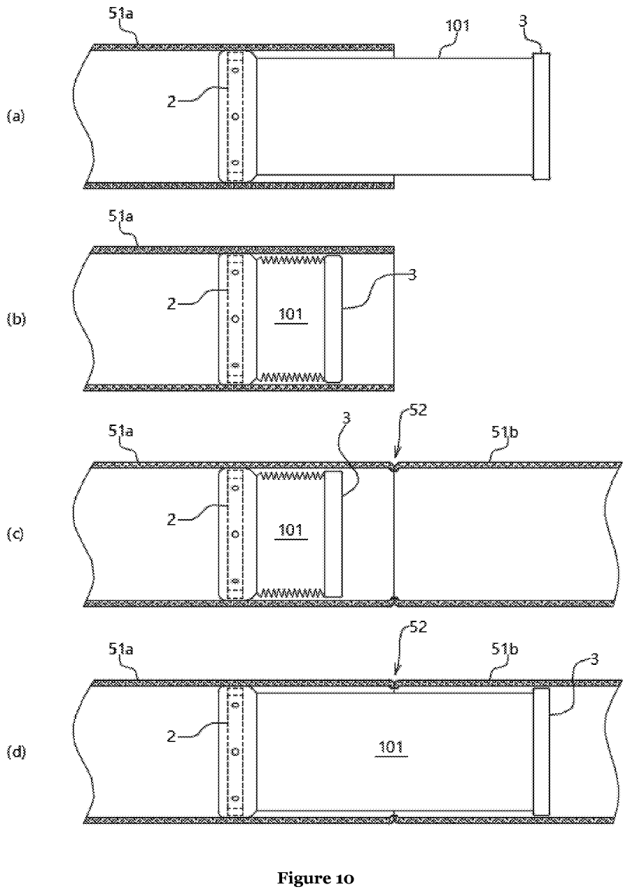

Referring to FIGS. 10(a) to (d), an exemplary usage process of the intestinal postoperative anti-fistula device provided by the present application is described as follows.
Before intestinal anastomosis, referring to FIG. 10(a), the first end of the anti-fistula device is placed into a proximal intestine 51a (e.g., the colon). The degradable proximal ring 2 is then sutured to the proximal intestine 51a using barbed sutures, thereby fixing both the degradable proximal ring 2 and the membrane tube 101 to the proximal intestine 51a.
Referring to FIG. 10(b), the membrane tube 101 is axially compressed, so that the entire anti-fistula device including the distal support ring 3 enters into the proximal intestine 51a.
Referring to FIG. 10(c), the proximal intestine 51a and a distal intestine 51b are anastomosed, forming an anastomosis 52. This procedure can be performed using an intestinal stapler, wherein the anvil of the stapler is placed in the proximal intestine 51a.
After the anastomosis, the distal support ring 3 is pulled towards the distal intestine 51b, using a medical instrument or hand externally from the intestine, such that the distal support ring 3 guides the membrane tube 101 to extend axially. Consequently, referring to FIG. 10(d), the membrane tube 101 completely covers the anastomosis, thereby isolating intestinal content from the anastomosis, preventing intestinal content from contacting the anastomosis, and fully protecting the anastomosis.

During the healing process of the anastomosis, microorganisms from the intestinal lumen enter the storage cavity 102 through the degradation through-holes 4 and come into contact with the circular degradable ring 201, causing the circular degradable ring 201 to gradually degrade. After the anastomosis heals completely, intestinal peristalsis and absorption of the sutures cause the membrane tube 101 to detach. The degradation products, remaining membrane tube 101, degradable proximal ring 2, and distal support ring 3 are excreted from the body with the stool, leaving no residue in the patient's body.

Using the anti-fistula device provided in Embodiment 1, a piglet experiment was conducted as follows:
1. A white pig was selected as an experimental animal. General anesthesia was administered with tracheal intubation.
2. After laparotomy, the sigmoid colon was selected for the experiment. The intestine was incised, feces were removed from the intestine, and thorough disinfection was performed. The anti-fistula device was placed through the intestinal incision. Then the anastomotic site was sutured. Barbed sutures were used for full-thickness and seromuscular layer suturing, with three fixation stitches placed along the circumferential direction.
3. After anastomosis completion, an image was obtained. The first end of the anti-fistula device (the end with the degradable proximal ring 2) was on the left side of the image, the second end of the anti-fistula device (the end with the distal support ring 3) was on the right side, and the anastomosis was in the center of the image.
4. Observation on the second postoperative day: the laparotomy wound healed well, with no exudate; and the pig ate and drank normally, urinated, and had no bowel movement yet.
5. Observation on the fourth postoperative day: unformed stool was passed; and the pig ate well and was in good condition.
6. Observation on the fifth postoperative day: formed stool was passed.
7. On the ninth postoperative day, laparotomy was performed to observe the condition of the anti-fistula device. The anti-fistula device was no longer present in the intestinal lumen, and it was presumed to have been excreted with the stool. A new anti-fistula device was placed, which was secured to prevent excretion with stool by adding a few more sutures.
8. On the tenth postoperative day, the initially placed anti-fistula device was found in the pig's stool.

In summary, after intestinal incision and anastomosis, and placement of the anti-fistula device, the pig ate and defecated normally, with no signs of obstruction, indicating good patency of the anti-fistula device. The anti-fistula device was fixed to the intestinal tract with three stitches, which was not secure enough. More stitches can be added to increase stability.

Using the anti-fistula device provided in Embodiment 2, a piglet experiment was conducted as follows:
1. Similarly, a white pig was selected as an experimental animal, and general anesthesia was administered with tracheal intubation.
2. After laparotomy, intestinal transection was performed. The anti-fistula device was placed 1 centimeter (cm) from the anastomotic site. Barbed sutures were used to fix the degradable proximal ring 2 and the membrane tube 101. 3-0 absorbable sutures were used for full-thickness suturing to partially close the anastomotic site, leaving a 1 cm deliberate leak.
3. To ensure complete suturing and fixation of the proximal ring to the intestine, the intestine was everted to find any area that was not tightly closed. 3-0 absorbable sutures were used to close any found opening, making the suturing more secure.
4. Postoperative days 3-5: stool passage was smooth, intake of food and water was normal, mental state and general condition were good; and the anti-fistula device was not excreted.
5. On the 13th postoperative day, the anti-fistula device was excreted with stool.

In summary, the anti-fistula device can effectively prevent the feces from contacting the anastomosis. Even in the presence of a leak, it can fully protect the anastomosis and promote anastomotic healing. Furthermore, its patency is good.

The above are only some specific embodiments of the present application, and the protection scope of the present invention is not limited thereto. Any person skilled in the art can easily conceive of various changes or substitutions within the technical scope disclosed by the present application, and these should be covered within the protection scope of the present application. The protection scope of the present application should be subject to the protection scope defined by the claims.

1. An intestinal postoperative anti-fistula device comprising an anti-fistula membrane assembly and a degradable proximal ring, the anti-fistula membrane assembly comprising a membrane tube, wherein:
a first end of the membrane tube is provided with a storage cavity formed by a folding section at the first end of the membrane tube, the degradable proximal ring is arranged in the storage cavity and configured to fix the membrane tube to an intestine by suturing, and a first wall of the storage cavity is provided with a plurality of degradation through-holes uniformly distributed; and
a second end of the membrane tube away from the storage cavity is provided with a distal support ring configured to provide support for the second end and guide the membrane tube to axially compress and extend, the distal support ring comprises three first segments and three second segments arranged alternately and connected end-to-end to form a complete circular ring, the second end is fixedly connected to one side of the three first segments and the three second segments, a thickness of each first segment is 2 mm, and a thickness of each second segment is 1 mm.

2. The intestinal postoperative anti-fistula device of claim 1, wherein the degradable proximal ring is a circular degradable ring hemmed in by walls of the storage cavity, an outer diameter of the circular degradable ring is 33 mm, and a width of the circular degradable ring is 3 mm.

3. The intestinal postoperative anti-fistula device of claim 1, wherein the degradable proximal ring is a wave-shaped degradable ring hemmed in by walls of the storage cavity, an outer diameter of the wave-shaped degradable ring is 33 mm, a maximum width of the wave-shaped degradable ring is 3.5 mm, and a minimum width of the wave-shaped degradable ring is 3 mm.

4. The intestinal postoperative anti-fistula device of claim 1, wherein the membrane tube and the degradable proximal ring are made of polyglycolic acid (PGA) and barium sulfate composite material with elasticity.

5. The intestinal postoperative anti-fistula device of claim 4, wherein the degradable proximal ring is a circular degradable ring hemmed in by walls of the storage cavity, an outer diameter of the circular degradable ring is 33 mm, and a width of the circular degradable ring is 3 mm.

6. The intestinal postoperative anti-fistula device of claim 4, wherein the degradable proximal ring is a wave-shaped degradable ring hemmed in by walls of the storage cavity, an outer diameter of the wave-shaped degradable ring is 33 mm, a maximum width of the wave-shaped degradable ring is 3.5 mm, and a minimum width of the wave-shaped degradable ring is 3 mm.

7. The intestinal postoperative anti-fistula device of claim 4, wherein a degradation and detachment time of the anti-fistula membrane assembly and the degradable proximal ring is 10 to 14 days, and at least one surface of the membrane tube is coated with lubricant.

8. The intestinal postoperative anti-fistula device of claim 7, wherein the degradable proximal ring is a circular degradable ring hemmed in by walls of the storage cavity, an outer diameter of the circular degradable ring is 33 mm, and a width of the circular degradable ring is 3 mm.

9. The intestinal postoperative anti-fistula device of claim 7, wherein the degradable proximal ring is a wave-shaped degradable ring hemmed in by walls of the storage cavity, an outer diameter of the wave-shaped degradable ring is 33 mm, a maximum width of the wave-shaped degradable ring is 3.5 mm, and a minimum width of the wave-shaped degradable ring is 3 mm.

10. The intestinal postoperative anti-fistula device of claim 1, wherein the degradable proximal ring is configured to be sutured with the intestine using barbed suture.

11. The intestinal postoperative anti-fistula device of claim 10, wherein the degradable proximal ring is a circular degradable ring hemmed in by walls of the storage cavity, an outer diameter of the circular degradable ring is 33 mm, and a width of the circular degradable ring is 3 mm.

12. The intestinal postoperative anti-fistula device of claim 10, wherein the degradable proximal ring is a wave-shaped degradable ring hemmed in by walls of the storage cavity, an outer diameter of the wave-shaped degradable ring is 33 mm, a maximum width of the wave-shaped degradable ring is 3.5 mm, and a minimum width of the wave-shaped degradable ring is 3 mm.

13. The intestinal postoperative anti-fistula device of claim 1, wherein the intestinal postoperative anti-fistula device is applicable for fistula prevention following sphincter-preserving surgery for low or ultra-low rectal cancer.
